(19) United States
(12) Patent Application Publication
(10) Pub. No.: US 2006/0205771 A1
(43) Pub. Date: Sep. 14, 2006

(54) Caspase inhibitors as anticancer agents

(76) Inventors: Mark Noble, Rochester, NY (US); Joerg Dietrich, Boston, MA (US)

Correspondence Address: Edwin V Merkel;Nixon Peabody, Clinton Square, P O Box 31051, Rochester, NY, 14603, US

(21) Appl. No.: 10/529,314

(22) PCT Filed: Sep. 25, 2003
(86) PCT No.: PCT/US03/30607

§ 371 Date: Apr. 11, 2006

Related U.S. Application Data
(60) Provisional application No. 60/413,409, filed on Sep. 25, 2002.

(51) Int. Cl. A61K 38/16; A61K 31/47; A61K 38/44; A61K 31/355; A61K 31/7048; A61K 31/375; A61K 31/353; A61K 38/17; A61K 38/05

(52) U.S. Cl. 514/310; 514/18; 424/94.4; 514/458; 514/474; 514/6; 514/763; 514/27; 514/456

(57) ABSTRACT

Disclosed are compositions and methods for treating cancer that contain caspase inhibitors and/or antioxidant compositions. Formulations containing noncaspase inhibitor anti-cancer agents are also disclosed.

### I. BACKGROUND OF THE INVENTION

One of the greatest needs in the field of cancer treatment is to develop means of selectively enhancing the killing of cancer cells. The most frequently used treatment methods for cancer patients, exposure to radiation and/or chemotherapy, kill many normal cells as effectively—or even more effectively—than they kill cancer cells. Thus, dose-limiting toxicity of these treatments is a serious issue. Side effects of chemotherapy are numerous, and range from the relatively benign loss of hair to myelotoxicity, cognitive impairment, liver and kidney damage, heart damage and damage to multiple other organ systems, as examples. There is a need for anti-cancer interventions that reduce the toxicity to non-cancer cells and for reagents that enhance the killing of cancer cells by any anti-cancer treatment which also can harm non-cancer cells without enhancing the killing of normal cells. Disclosed are compositions and methods that address these needs. Disclosed are compositions and methods that inhibit cancer cell growth without increasing non-cancer cell toxicity. Also disclosed are compositions and methods that work in combination with any other anti-cancer regimen, wherein the overall cellular toxicity to normal cells is reduced because a similar amount of anti-cancer activity is seen with a reduced amount of the anti-cancer regimen which is toxic to non-cancer cells.

### II. SUMMARY OF THE INVENTION

In accordance with the purposes of this invention, as embodied and broadly described herein, this invention, in one aspect, relates to anti-cancer reagents.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### III. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows that the exposure of the 1789 glioblastoma cell line to a pan-caspase inhibitor causes a reduction in cell number equivalent to the effects of exposure to BCNU. Combined exposure to BCNU and the pan-caspase inhibitor significantly increased the amount of cell death over that caused by exposure to BCNU alone. A similar enhancement of BCNU-induced killing was caused by co-exposure to BCNU and an inhibitor of caspase 3.

FIG. 2 shows that the exposure of the 1789 glioblastoma cell line to an inhibitor of caspase-9 causes a reduction in cell number equivalent to the effects of exposure to BCNU alone. Combined exposure to BCNU and an inhibitor of caspase-9 significantly increased the amount of cell death over that caused by exposure to BCNU alone.

FIG. 3 shows that the exposure of the UT-12 glioblastoma cell line to an inhibitor of caspase-9 causes a reduction in cell number even greater than the effects of exposure to BCNU. Similar reductions are caused by exposure to a combination of caspase-8 and caspase-9 inhibitors. Combined exposure to BCNU and inhibitors of caspase-8 and caspase-9 applied together with BCNU was associated with significantly increased cell death over that caused by exposure to BCNU alone. In addition, the combination of BCNU and inhibitors of caspase-8 and -9 caused a significantly greater killing of cancer cells than did application of the caspase inhibitors by themselves or by the application of BCNU by itself.

FIG. 4 shows that the combined exposure of the UT-12 glioblastoma cell line to BCNU and a pan-caspase inhibitor significantly increased the amount of cell death over that caused by exposure to BCNU alone.

FIG. 5 shows that the exposure of the UT-9 astrocytoma cell line (derived from a low grade astrocytoma, WHO grade 11) to BCNU (at equivalent doses used for the glioblastoma cell lines 1789 and UT-12) causes only a minor reduction in cell number. In contrast when BCNU is added together with an inhibitor of caspase-9 the number of cells killed is significantly increased. These experiments suggest that caspase inhibitor activation may also be able to overcome chemoresistance.

FIG. 6 shows the cytotoxic effect of caspase 9 and pan-caspase inhibition in combination with BCNU can be further enhanced by application of Vitamin C. The fill combination kills all of the UT-12 glioma cells. Thus, caspase inhibitors may be applied in combination with other non-toxic compounds to further enhance chemosensitivity in cancer cells.

FIG. 7 shows that in contrast to the effects of caspase inhibitors in enhancing the killing of tumor cells (as shown in FIG. 1-6), these same inhibitors do not have such effects on normal human brain precursor cells. This example shows treatment of human glia restricted precursor cells (GRP) with BCNU. Caspase 8 and 9 inhibitors do not enhance the cytotoxic activity of BCNU nor do they compromise the viability of human GRP cells when applied by themselves.

FIG. 8 shows that caspase inhibitors do not enhance the cytotoxic effects of BCNU on normal astrocytes. Astrocytes were killed by BCNU but not by inhibitor of caspase-8. While inhibition of caspase-8 did not rescue these cells, neither did it make them worse than BCNU alone. The failure to rescue is consistent with ideas that BCNU might preferentially work through activation of caspase-9. In support of this, inhibition of caspase-9 actually conferred partial protection on astrocytes.

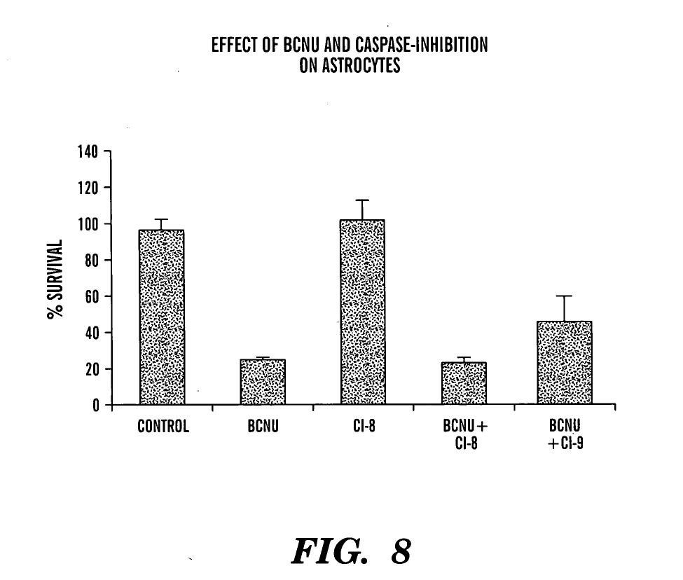

FIG. 9 shows that co-application of a caspase inhibitor with an anti-oxidant is more effective at killing tumor cells than application of the caspase inhibitor by itself.

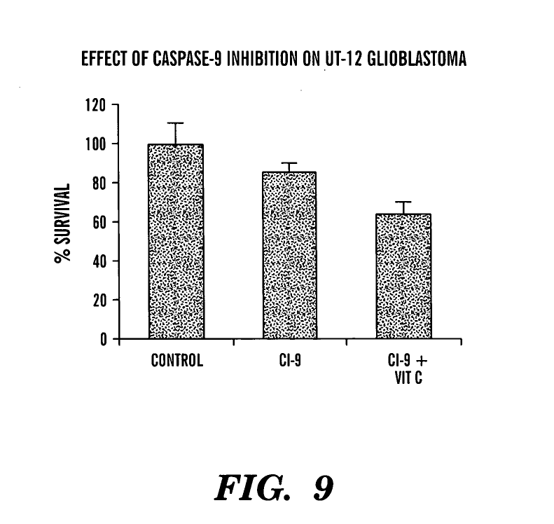

FIG. 10 shows that application of caspase inhibitors to SW480 colon cancer cells not only fails to rescue from cisplatin-induced death, but actually decreases the number of cells still further from the reduction obtained with cisplatin alone.

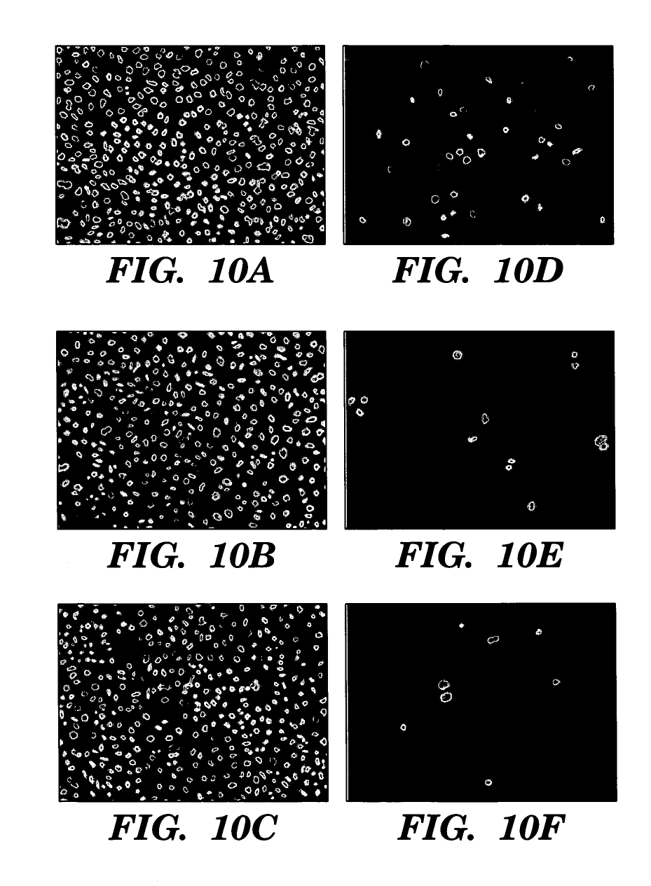

### IV. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

### B. COMPOSITIONS AND METHODS

Disclosed are compositions and methods for treating cancers in subjects. The disclosed compositions and methods are capable of inhibiting uncontrolled cellular proliferation or aberrant cellular proliferation. The disclosed compositions and methods are useful for slowing the growth of cancerous cells. The disclosed compositions and methods are useful for slowing the spread of cancerous cells.

The disclosed compositions represent either caspase inhibitors, combinations of caspase inhibitors, or, more typically mixtures of compositions, of which caspase inhibitors represent one component. The mixtures typically include an anticancer agent or mixtures of anti-cancer agents, such as an antimetabolite, an alkylating agent, a topoisomerase inhibitor or other anti-cancer agent, applied in combination with a caspase inhibitor or mixtures of caspase inhibitors. These mixtures can be used in the disclosed methods, for example, to treat cancer. The mixtures can also include anti-oxidants in any combination, such as in the combination of caspase inhibitors and anti-oxidants.

The disclosed compositions comprise caspase inhibitors. Caspase inhibitors are reagents that inhibit caspase activity. Capsases are a family of proteins that are involved in apoptotic cell death.

Anti-cancer treatments are typically designed to induce or accelerate cell death. There are different types of cell death pathways, however, which rely on different cell signaling pathways and utilize different sets of enzymes. Thus, compositions that can induce the activation of one path are not necessarily able to target another path. There are at least three broad types of cell death: necrosis, apoptosis and parapoptosis, and caspases are involved in both apoptosis and parapoptosis. For general reviews, one may consult Wang and Lenardo, 2000, Journal of Cell Science 113, 753-757; Budihardjo et al., Annu. Rev. Cell Dev. Biol. 1999. 15:269-90; Sperandio et al., 2000, Proc. Natl. Acad. Sci., USA, 97:14376 and references included therein, as examples).

Necrosis is a form a cell death that does not require gene expression. It is characterized, among other aspects, by cytoplasmic vacuolation and mitochondrial swelling, but not by nuclear fragmentation and chromatin condensation. Internucleosomal DNA fragmentation is not observed, and TUNNEL staining is usually not observed. In relation to caspase activity, it appears that DEVD-cleaving activity is not important nor is caspase-3 processing. PARP cleavage occurs to 50-62 kDA fragments occurs (as contrasted with cleavage to the 85-kDa fragment that occurs in apoptosis). There is no inhibition by such reagents as zVAD.fmk, BAF, p35, xiap, and generally not by Bcl-x_{L}. There is no inhibition by actinomycin D or cycloheximide.

Apoptosis is a form a cell death that requires gene expression. It is characterized, among other aspects by nuclear fragmentation and chromatin condensation. Mitochondrial swelling may or may not occur. Internucleosomal DNA fragmentation is observed, as is TUNEL staining. In relation to caspase activity, DEVD-cleaving activity is important as is caspase-3 processing and PARP cleavage. There is inhibition by such reagents as zVAD.fmk, BAF, p35, xiap, Bcl-x_{L}. There may be inhibition by actinomycin D or cycloheximide, working as inhibitors of gene expression.

Parapoptosis is a form of nonapoptotic programmed cell death that fails to fulfill the requirements for apoptosis (Sperandio S, de Belle I, Bredesen DE. An alternative, nonapoptotic form of programmed cell death Proc Natl Acad Sci. USA. Dec. 19, 2000;97(26):14376-81). This type of cell death is not inhibited by caspase inhibitors or by BCI-x_{L} but is inhibited by a catalytic mutant of caspase-9 zymogen. The parapoptosis pathway mediated by caspase-9 is Apaf-1 independent and is not inhibited by mutation of the sites of zymogen process to the nonapoptotically active forms. It is not characterized by nuclear fragmentation, although there may be some chromatin condensation (but less so than for apoptosis). Mitochondrial swelling occurs late, and cytoplasmic vacuolation does occur. Internucleosomal DNA fragmentation is not observed, nor is TUNEL staining. In relation to caspase activity, it is clear that DEVD-cleaving activity is not important, nor is caspase-3 processing or PARP cleavage. There is no inhibition by such reagents as zVAD.fmk, BAF, p35, xiap, BCl-x_{L}. There is inhibition, in contrast, by actinomycin D or cycloheximide.

Anti-cancer regimens can target these various pathways to effect the death or inhibition of cancer cells. For example, if a particular type of cancer cell is predisposed to die via one pathway, reagents to target that cancer cell can target the activation of that particular pathway. Likewise if non-cancer cells are predisposed to, one type of cell death, then reagents that activate that pathway, even if they kill cancer cells also, would not be preferred because of their lack of specificity for cancer cells.

1. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective permutation of these compounds may not be explicitly disclosed, each is specifically contemplated and described herein. For example, if a particular caspase inhibitor is disclosed and discussed and a number of modifications that can be made to a number of molecules including the caspase inhibitor are discussed, specifically contemplated is each and every combination and permutation of caspase inhibitors and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated, meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Disclosed herein is that the inhibition of caspase function in cancer cells can induce death of these cells. Also disclosed is that caspase inhibition can enhance death of cancer cells induced by treatment regimens used in cancer patients. As the caspase inhibitors have no apparent cytotoxic activity on normal (non-transformed cells), and indeed can rescue normal (non-transformed) cells from the cytotoxic effects of chemotherapeutic agents, they can be used as anticancer agents alone or as additions to other anti-cancer regimens to selectively enhance the killing of cancer cells.

a) Caspases

The process of cell death has been enhanced by the discovery of the caspases, also known as interleukin-converting enzymes (ICE) and zymogens. The caspases are a family of cysteine proteases that act in a cascade to trigger the process of apoptosis. These results indicate that caspase activation is critical to the function of a transformed cell. Thus, identifying other means of preventing such activation other then, for example, direct caspase inhibition, would be expected to have similar therapeutic benefit. Caspases-3, -6 and -7 are involved in the execution of cells in response to a variety of apoptotic inducers, such as activation of death receptors of the tumor necrosis receptor-1 family. These execution caspases are not directly activated by receptor activation, but instead are activated by the proteolytic activity of an upstream initiator, such as caspases-8 and -10. Typically caspase-3 is upstream of capases-6 and -7, and caspase-8 is upstream of caspase-3. Caspase-8 can also activate caspase-9. Caspase-9 can also be activated by pro-apoptotic stimuli other than activating death receptors. Caspase-9 activation, in turn, can lead to activation of the execution caspases, as well as to activation of caspase-8 and caspase-10. Caspase-9 can itself also be activated by a separate mechanism, leading to induction of parapoptosis.

Multiple biochemical pathways can contribute to cell death, and numerous cell death pathways involving caspases, enzymes that play important roles in the initiation of apoptosis or other forms of cell death, have been identified. For general reviews, one may consult Wang and Lenardo, 2000, Journal of Cell Science 113, 753-757; Budihardjo et al., Annu. Rev. Cell Dev. Biol. 1999. 15:269-90; Sperandio et al., 2000, Proc. Natl. Acad. Sci., USA, 97:14376 and references included therein, as examples). Even within the family of caspases, different caspases can be central in different types of cell death and/or in different cell populations. Cancer cells can differ from normal cells not only in the metabolic balances that are able to initiate cell death, but also in effector pathways that are utilized in the death process. This is indicated by studies demonstrating that the tumor necrosis factor-related apoptosis-inducing ligand (TRAIL) induces cell death in human liver cells by a caspase-9 dependent mechanism. The caspase 9-inhibitor Z-LEHD-FMK effectively protects liver cells from TRAIL-associated toxicity. In contrast, this inhibitor did not protect SW480 (colon adenocarcinoma) and H460 (non-small cell lung cancer) cell lines from TRAIL induced death. Typically caspases 8 and 9 are differentially regulated, with caspase 8 being cleaved by Fas-related pathways and caspase 9 being cleaved through a broader range of apoptotic stimuli (including as a consequence of caspase 8 activation) (e.g., Budihardjo, I., Oliver, H., Lutter, M., Luo, X., and Wang, X. Biochemical pathways of caspase activation during apoptosis., Annu. Rev. Cell Dev. Biol. 15: 269-290, 1999 and Kruidering, M. and Evan, G. I. Caspase-8 in apoptosis: the beginning of “the end.”, IUBMB Life. 50: 85-90, 2000 for review).

(1) Caspase Inhibitors

Knowledge of the caspase structures, and recognition of their importance, has led to the development of a wide variety of caspase inhibitors. It has been possible to develop pan-caspase inhibitors that stop activation of all caspases, as well as specific inhibitors of individual caspases. In particular, specific inhibitors exist for caspases 8, 9, 3, and 1. Several peptide-based inhibitors have been designed, mainly tetrapeptide-inhibitors (as described, for example, in Cryns and Yuan, 1998, Genes Dev. 12:1551: Talanian et al., 1997, J. Biol. Chem. 272:9677; Garcia-Calvo et al., 1998, J. Biol. Chem. 273:32608. The peptide sequences are based on the recognition sequence of substrates, which are cleaved by particular caspases. For example, the tetrapeptide aldehyde Ac-YVAD-CHO is based on the pro-IL-1beta cleavage site, and therefore is a strong inhibitor of Caspase-1, while the aldehyde tetrapeptide containing the PARP cleavage-site, c-DEVD-CHO, inhibits preferentially (but not specifically) caspase-3. Peptide based inhibitors are available for all caspases, as indicated from example the Caspase inhibitor Sample Pack Catalog number FMKSP01 from R&D Systems (published Mar. 13, 2000), Caspase inhibitor literature from Pharmingen, Inc. and multiple other companies well known to skilled practicioners of the arts relevant to this invention. The peptide z-VAD-fink is a broad-range caspase inhibitor. Typically caspase inhibitors are characterized by their ability to interfere in the process of cell death by apoptosis. Caspase inhibitors have been documented at preventing cell death in normal cells and in tumor cell lines, as described for example in such references as (Schlegel et al., 1996, J. Biol. Chem., 271:1841; Martins et al., 1997, J. Biol. Chem. 272:7421; Huany et al., 1999, Mol. Cell. Biol. 19:2986; Guo and Kyprianou, 1999, Cancer Res. 59:1366; Ulaisincharoen et al., 1999, Clin. Exp. Immunol. 116:41; Zaks et al, 1999, J. Immunol. 162:3273; Gastman et al., 1999, Cancer Res. 59:1422). Thus, there is extensive examination of the potency of these caspase inhibitors.

Caspase activation has also been indicated in the proliferation of pro-T cells, and it appears that caspases are activated in primary T-cells after anti-CD3 stimulation and that this activation is necessary for the proliferative response. It has been indicated that NIH3T3 cells are sensitized to the action of tumor necrosis factors and other death inducing ligands by inhibition of Fas-associated death domain protein/caspase-8 signaling. Cells show an accumulation in the G2/M phase of the cell cycle, but die instead of further advancing, showing several features of apoptosis despite the lack of caspase-3 activity. Sensitization to the action of TNF was associated also with exposure to NIH3T3 cells to zVAD.fmk, a broad spectrum caspase inhibitor (Luschen et al, 2000, J. Biol. Chem. 275:24670). In regards to the present application it is important that data is interpreted in terms of specific interactions with death domain receptors and their adapter proteins, such as the Fas-associated death domain protein.

In contrast with the above work, it has been shown that apoptosis induced by death receptor-triggering is blocked by overexpressing dominant negative forms of FADD or caspase-8 or by inhibiting either caspases or, in some cells, mitochondrial cytochrome c release (Hsu, H., Shu, H. B., Pan, M. G., and Goeddel, D. V. (1996) Cell 84, 299-308; Boldin, M. P., Goncharov, T. M., Goltsev, Y. V., and Wallach, D. (1996) Cell 85, 803-815; Wajant, H., Johannes, F. J., Haas, E., Siemienski, K., Schwenzer, R., Schubert, Y. G., Weiss, T., Grell, M., and Scheurich, P. (1998) Curr. Biol. 8, 113-116; Scaffidi, C., Fulda, S., Srinivasan, A., Friesen, C., Li, F., Tomaselli, K. J., Debatin, K.-M., Krammer, P. H., and Peter, M. E. (1998) EMBO J. 17, 1675-1687). Indeed, in the work by Luschen et al. it was observed that caspase-8 inhibition was not toxic for HELA cells and indeed protected these cells from TNF-induced apoptosis. It has been reported, though, that in the case of U937 cells caspase-8 inhibition may also increase sensitivity to tumor necrosis factor (Khwaja, A., and Tatton, L. (1999) J. Biol. Chem. 274, 36817-36823). It is important to note in these regards, however, that despite its name, tumor necrosis factor is itself cytotoxic for only a minority of cancer cells. It appears that the enhancement of TNF-mediated death by caspase-8 inhibition in NIH3T3 cells and U937 cells have been considered to be special cases not revealing of general principles. In contrast, in the instant invention, attention has been paid to very different classes of cytotoxic agents, these being chemotherapeutic agents. Moreover, the examples provided in the instant invention demonstrate the extension of the general principles we have discovered to also include inhibition of caspase-3 and caspase-9, in contrast with the studies of Luschen and others. Moreover, in the studies of Luschen et al., pretreatment with the radical scavenger butylated hydroxyanisole (BHA) protected NIH3T3 cells from cytotoxicity induced by the combination of tumor necrosis factor and caspase-8 inhibition, while the examples of the instant invention show that the combination of caspase-8 inhibition and anti-oxidant application is itself toxic for cancer cells.

There are many different caspase inhibitors which can be used in the disclosed methods and in conjunction with other non-caspase inhibitor anti-cancer agents. Many publications and patents provide detailed summaries of the wide variety of inhibitors, which may be peptide based or may be small molecule inhibitors, the following of which are exemplary and are herein incorporated by reference for material related to inhibition of capsases. A non-inclusive listing, which is not intended to be limited, demonstrating the diversity of approaches to the generation of caspase inhibitors, which would be of equal relevance to the contents of this invention is as follows:

U.S. Pat. No. 6,197,750 (Karanewsky, et al.) describes C-terminal modified oxamyl dipeptides as inhibitors of the ICE/ced-3 family of cysteine proteases.

U.S. Pat. No. 6,242,422 (Karanewsky, et al.) describes (substituted)Acyl dipeptidyl inhibitors: of the ice/ced-3 family of cysteine proteases

U.S. Pat. No. 6,187,771 (Karanewsky, et al.) describes tricyclic compounds for the inhibition of the ICE/ced-3 protease family of enzymes The compounds of this invention incorporate a conformationally constrained dipeptide mimetic. This mimetic exhibits improved properties relative to their peptidic counterparts, for example, such as improved absorption and stability resulting in enhanced bioavailability.

Examples of dipeptide inhibitors of caspases are descred in U.S. Pat. No. 6,184,244 (Karanewsky, et al.), which describes C-terminal modified (N-substituted)-2-indolyl dipeptides as inhibitors of the ICE/ced-3 family of cysteine proteases.

Other examples of modified dipeptide inhibitors of caspases are provided in U.S. Pat. No. 6,225,288 (Han, et al.), which describes gamma-ketoacid dipeptides as inhibitors of caspase-3.

Caspases can also be modulated by inhibiting their expression, for example by use of antisense compounds to specifically degrade the RNA encoding for specific caspases. Examples of such an approach to modulating caspase activity by modulating caspase expression itself are provided in U.S. Pat. No. 6,303,374 (Zhang, et al.), which describes antisense modulation of caspase 3 expression and U.S. Pat. No. 6,258,600 (Zhang; et al.), which describes antisense modulation of caspase 8 expression.

In addition to the inhibition of caspase activity, similar results would be expected to be obtained by reducing caspase expression. Such reduction in expression could be achieved by using, for example, technnologies that disrupt mRNA expression or function. Such technologies include anti-sense RNA (both catalytic and non-catalytic), RNA inhibition, direct inhibition of expression from caspase promoters, and other such approaches as will be apparent to skilled practicioners of the art.

(2) Non-Caspase Inhibitor Anticancer Agents

The disclosed compositions and methods of using the compositions can include the use of anti-cancer agents which are not also, caspase inhibitors. As disclosed herein, the combination of compositions which are caspase inhibitors with compositions that are not caspase inhibitors but are anti-cancer agents can have desirable anti-cancer activities. Any anti-cancer agent can be included in the disclosed compositions and used in the disclosed methods. The reference to non-caspase anticancer agents is not meant to indicate that caspase inhibitors are not anti-cancer agents as disclosed herein; caspase inhibitors also can act as anti-cancer agents. Rather, non-caspase inhibitor anti-cancer agents refers to compositions which do not function as caspase inhibitors but do have anti-cancer activity. Typically non-caspase inhibitor anti-cancer agents will affect the death of non-cancer cells as well as cancer cells, but the non-caspase inhibitor anti-cancer agents used in the present compositions and methods need not have this effect. Thus, typically non-caspase anti-cancer agents are toxic to non-cancer cells.

Numerous types of anti-cancer agents exist which are understood to not also have caspase inhibitor activity. For example, non-caspase inhibitor anti-cancer agents can include, for example, DNA interactive agents, such as DNA intercalating agents, DNA alkylating agents, and DNA strand breaking agents, DNA topoisomerase II inhibitors, antimetabolites, and tubulin interactive agents.

A non-limiting list of DNA-interactive agents includes the alkylating agents, such as Cisplatin, Cyclophosphamide, Altretamine; the DNA strand-breakage agents, such as Bleomycin; and the intercalating topoisomerase II inhibitors, such as Dactinomycin and Doxorubicin; the nonintercalating topoisomerase II inhibitors such as, Etoposide and Teniposide; and the DNA minor groove binder Plicamycin.

DNA alkylating agents form covalent chemical adducts with cellular DNA, RNA, protein molecules, smaller amino acids, glutathione, and similar chemicals. Generally, these alkylating agents react with a nucleophilic atom in a cellular constituent, such as an amino, carboxyl, phosphate, sulfhydryl group in nucleic acids, proteins, amino acids, or glutathione.

Typical alkylating agents include: Nitrogen mustards, such as Chlorambucil, Cyclophosphamide, Isofamide, Mechlorethamine, Melphalan, Uracil mustard; aziridines such as Thiotepa; ïmethanesulfonate esters such as Busulfan; nitroso ureas, such as Carmustine, Lomustine, Streptozocin; platinum complexes, such as Cisplatin, Carboplatin; ïbioreductive alkylator, such as Mitomycin, and Procarbazine, Dacarbazine and Altretamine.

A non-limiting DNA topoisomerase II inhibitor list includes: Intercalators such as Amsacrine, Dactinomycin, Daunorubicin, Doxorubicin, Idarubicin, and Mitoxantrone; ïnonintercalators, such as Etoposide and Teniposide. The antimetabolites interfere with the production of nucleic acids typically by one or the other of two major mechanisms. First, some of the antimetabolites inhibit production of the deoxyribonucleoside triphosphates that are the immediate precursors for DNA synthesis, thus inhibiting DNA replication. Second, some of the antimetabolites are sufficiently like purines or pyrimidines to be able to substitute for them in the anabolic nucleotide pathways. These analogs can then be substituted into the DNA and RNA instead of their normal counterparts. Exemplary antimetabolites useful herein include: folate antagonists such as Methotrexate and trimetrexate pyrimidine antagonists, such as Fluorouracil, Fluorodeoxyuridine, CB3717, Azacytidine, Cytarabine, and Floxuridine purine antagonists, which include Mercaptopurine, 6-Thioguanine, Fludarabine, Pentostatin; sugar modified analogs, which include Cyctrabine, Fludarabine; and Ribonucleotide reductase inhibitors, which include hydroxyurea.

Farnesyltransferase inhibitors are also useful anti-cancer agents. Farnesyltransferase inhibitors are used to prevent farnesylation of signaling molecules thus preventing their necessary integration into the cell membrane. Multiple farnesyltransferase inhibitors have been identified, for example as described in U.S. Pat. No. 6,218,406.

Tubulin interactive agents are also useful anti-cancer agents. Tubulin interactive agents act by binding to specific sites on tubulin, a protein that polymerizes to form cellular microtubules. Microtubules are critical cell structure units. When the interactive agents bind on the protein, the cell cannot form microtubules Tubulin interactive agents include Vincristine and Vinblastine, both alkaloids and Paclitaxel.

Adrenal corticosteroids are also considered useful anti-cancer agents. Adrenal corticosteroids are derived from natural adrenal cortisol or hydrocortisone. They are used because of their anti inflammatory benefits as well as the ability of some to inhibit mitotic divisions and to halt DNA synthesis. These compounds include, Prednisone, Dexamethasone, Methylprednisolone, and Prednisolone.

Other anti-cancer agents can include, for example, the following. Hydroxyurea appears to act primarily through inhibition of the enzyme ribonucleotide reductase. Asparagenase is an enzyme which converts asparagine to nonfunctional aspartic acid and thus blocks protein synthesis in the tumor. The hormonal agents and leutinizing hormones are not usually used to substantially reduce the tumor mass. However, they can be used in conjunction with the chemotherapuetic agents or the benzimidazoles.

Hormonal blocking agents are also useful in the treatment of cancers and tumors. They are used in hormonally susceptible tumors and are usually derived from natural sources. These include: estrogens, conjugated estrogens and Ethinyl Estradiol and Diethylstilbesterol, Chlorotrianisene and Idenestrol; progestins such as Hydroxyprogesterone caproate, Medroxyprogesterone, and Megestrol; androgens such as testosterone, testosterone propionate; fluoxymesterone, methyltestosterone; Leutinizing hormone releasing hormone agents or gonadotropin-releasing hormone antagonists are used primarily in the treatment of prostate cancer. These include leuprolide acetate and goserelin acetate. They prevent the biosynthesis of steroids in the testes.

Antihormonal agents include: antiestrogenic agents such as Tamoxifen, ïantiandrogen agents such as Flutamide; and antiadrenal agents such as Mitotane and Aminoglutethimide.

Still another class of potential antitumor agents are the general class of inhibitors of cyclin dependent kinases. Examples of such compounds include the aminothiazole inhibitors described in U.S. Pat. No. 6,262,096.

Novel alkyl ketone compounds having potent cytotoxic activity have been described (U.S. Pat. No. 6,251,882 incorporated herein by reference at least for material related to anti-cancer compounds and alkyl ketone compounds) as anti-tumor agents and are particularly effective against leukemia and breast tumor cells. Inhibitors of signaling molecules, such as Glivec, tyrphostins and other such inhibitors that interrupt the cascade of signaling events involved in cell division and/or cell survival represent still another example of cancer treatment agents.

It is understood that these are representative compositions and that the anti-cancer agents are not limited to these unless so indicated. Furthermore, it is understood that each of the disclosed anti-cancer compositions disclosed herein is also individually disclosed herein.

(3) Antioxidants

Antioxidants have also been shown to have antitumor activity and can be used in any combination in the disclosed mixtures.

Generally, antioxidants are compounds that react with oxygen and reactive oxidative intermediates. Since antioxidants typically react with oxygen, antioxidants also typically react with the free radical generators, and free radicals. (“The Antioxidants—The Nutrients that Guard Your Body” by Richard A. Passwater, Ph.D., 1985, Keats Publishing Inc., which is herein incorporated by reference at least for material related to antioxidants). The compositions can contain any antioxidants, and a non-limiting list would included but not be limited to, non-flavonoid antioxidants and nutrients that can directly scavenge free radicals including multi-carotenes, beta-carotenes, alpha-carotenes, gamma-carotenes, lycopene, lutein and zeanthins, selenium, Vitamin E, including alpha-, beta- and gamma-(tocopherol, particularly .alpha.-tocopherol, etc., vitamin E succinate, and trolox (a soluble Vitamin E analog) Vitamin C (ascoribic acid) and Niacin (Vitamin B3, nicotinic acid and nicotinamide), Vitamin A, 13-cis retinoic acid, N-acetyl-L-cysteine (NAC) and other glutathione pro-drugs, sodium ascorbate, pyrrolidin-edithio-carbamate, and coenzyme Q10; enzymes which catalyze the destruction of free radicals including peroxidases such as glutathione peroxidase (GSHPX) which acts on H₂O₂ and such as organic peroxides, including catalase (CAT) which acts on H₂O₂, superoxide dismutase (SOD) which disproportionates O₂H₂O₂; glutathione transferase (GSHTx), glutathione reductase (GR), glucose 6-phosphate dehydrogenase (G6PD), and mimetics, analogs and polymers thereof (analogs and polymers of antioxidant enzymes, such as SOD, are described in, for example, U.S. Pat. No. 5,171,680 which is incorporated herein by reference for material at least related to antioxidants and antioxidant enzymes); glutathione; ceruloplasmin; cysteine, and cysteamine (beta-mercaptoethylamine) and flavenoids and flavenoid like molecules like folic acid and folate and spin-trap protectors against damage by reactive oxidative intermediates. A review of antioxidant enzymes and mimetics thereof and antioxidant nutrients can be found in Kumar et al, Pharmac. Ther. Vol 39: 301, 1988 and Machlin L. J. and Bendich, F.A.S.E.B. Journal Vol. 1:441-445, 1987 which are incorporated herein by reference for material related to antioxidants. In addition, redox potential of a cell can be manipulated through control of peroxisome function, which occurs through regulation of PPARs. Thus, as one embodiment of this invention, the combination of PPAR regulators chosen to promote a more reduced state in the cell (for example, PPAR-alpha antagonists or PPAR-gamma agonists) may be used in addition to, or in place of, more commonly used anti-oxidants.

Flavonoids, also known as “phenylchromones,” are naturally occurring, water-soluble compounds which have antioxidant characteristics. Flavonoids are widely distributed in vascular plants and are found in numerous vegetables, fruits and beverages such as tea and wine (particularly red wine). Flavonoids are conjugated aromatic compounds. The most widely occurring flavonoids are flavones and flavonols (for example, myricetin, (3,5,7,3′,4′,5′,-hexahydroxyflavone), quercetin (3,5,7,3′,4′-pentahydroxyflavone), kaempferol (3,5,7,4′-tetrahydroxyflavone), and flavones apigenin (5,7,4′-trihydroxyflavone) and luteolin (5,7,3′,4′-tetrahydroxyflavone) and glycosides thereof and quercetin).

b) Anti-Cancer Formulations

The disclosed compositions comprise caspase inhibitors. As disclosed herein, caspase inhibitors have anticancer activity when administered alone, but caspase inhibitors are also useful compounds to be administered in combination with other anti-cancer treatments, including in combination with chemotherapy treatments as well as radiological, surgical, and other cancer treatments. It is understood that the caspase inhibitors can be combined with non-caspase inhibitor anticancer agents and/or antioxidants. It is understood that the caspase inhibitors, non-caspase inhibitor anti-cancer agents, and antioxidants can also be used in any combination. For example, combinations of different antioxidants may be used in conjunction with one or more different caspase inhibitors. Likewise, combinations of different non-caspase inhibitor anti-cancer agents may be used in conjunction with one or more different caspase inhibitors. Furthermore, it is understood that the combinations disclosed herein can comprise any combination of caspase inhibitor.

(1) Caspase Inhibitors Alone

Disclosed herein are formulations of caspase inhibitors for administration to subjects needing anti-cancer treatment. As discussed herein any pharmaceutically acceptable carrier and formulation can be used. As disclosed herein, for anticancer therapeutic uses, the capase inhibitors have activity at the same concentrations for which they inhibit caspase activity, and for example, concentrations at which they inhibit apoptosis.

(2) Caspase Inhibitor+Non-Caspase Inhibitor Anti Cancer Agent Formulations

The disclosed compositions include mixtures of caspase inhibitors and other non-caspase inhibitor anti cancer agents. It is understood in the art that non-caspase inhibitor anti-cancer agents, typically can cause cell death to non-cancer cells as well as cancer cells. The toxic effect of non-caspase inhibitor anti-cancer agents can be reduced by the present mixtures of compositions because the present mixtures of compositions can provide similar levels of anti-cancer activity to the non-caspase inhibitor anti-cancer agent alone, even when the mixture contains a lower concentration of the non-caspase inhibitor anti-cancer agent as compared to a formulation containing the non-caspase inhibitor anti-cancer agent alone at a concentration that produces the level of anti-cancer activity.

Formulations of the caspase inhibitors can include concentrations at which caspase activity is inhibited and for example, where apoptosis is inhibited in non-cancerous cells. In addition, the formulations can include any therapeutic formulation of the non-caspase inhibitor anti-cancer agent(s). However, one of the benefits of the disclosed compositions and formulations is that the dose of the non-caspase inhibitor anti-cancer agent can be reduced while retaining the same level of therapeutic cancer cell killing if the non-caspase inhibitor anti-cancer is applied together with the caspase inhibitor.

One way of addressing this beneficial effect of the combination of the caspase inhibitor and the non-caspase inhibitor anti-cancer agent is to produce formulations that have at least about 99% or at least about 98% or at least about 97% or at least about 96% or at least about 95% or at least about 94% or at least about 93% or at least about 92% or at least about 91% or at least about 90% or at least about 89% or at least about 88% or at least about 87% or at least about 86% or at least about 85% or at least about 84% or at least about 83% or at least about 82% or at least about 81% or at least about 80% or at least about 79% or at least about 78% or at least about 77% or at least about 76% or at least about 75% or at least about 74% or at least about 73% or at least about 72% or at least about 71% or at least about 70% or at least about 69% or at least about 68% or at least about 67% or at least about 66% or at least about 65% or at least about 64% or at least about 63% or at least about 62% or at least about 61% or at least about 60% or at least about 59% or at least about 58% or at least about 57% or at least about 56% or at least about 55% or at least about 54% or at least about 53% or at least about 52% or at least about 51% or at least about 50% or at least about 49% or at least about 48% or at least about 47% or at least about 46% or at least about 45% or at least about 44% or at least about 43% or at least about 42% or at least about 41% or at least about 40% or at least about 39% or at least about 38% or at least about 37% or at least about 36% or at least about 35% or at least about 34% or at least about 33% or at least about 32% or at least about 31% or at least about 30% or at least about 29% or at least about 28% or at least about 27% or at least about 26% or at least about 25% or at least about 24% or at least about 23% or at least about 22% or at least about 21% or at least about 20% or at least about 19% or at least about 18% or at least about 17% or at least about 16% or at least about 15% or at least about 14% or at least about 13% or at least about 12% or at least about 11% or at least about 10% or at least about 9% or at least about 8% or at least about 7% or at least about 6% or at least about 5% or at least about 4% or at least about 3% or at least about 2% or at least about 1% of the amount of non-caspase inhibitor anti-cancer agent that would be used alone.

One way of addressing this beneficial effect of the combination of the caspase inhibitor and the non-caspase inhibitor anti-cancer agent is to produce formulations that have an amount of the non-caspase inhibitor anti-cancer agent that if used alone would produce at least about 99% or at least about 98% or at least about 97% or at least about 96% or at least about 95% or at least about 94% or at least about 93% or at least about 92% or at least about 91% or at least about 90% or at least about 89% or at least about 88% or at least about 87% or at least about 86% or at least about 85% or at least about 84% or at least about 83% or at least about 82% or at least about 81% or at least about 80% or at least about 79% or at least about 78% or at least about 77% or at least about 76% or at least about 75% or at least about 74% or at least about 73% or at least about 72% or at least about 71% or at least about 70%, or at least about 69% or at least about 68% or at least about 67% or at least about 66% or at least about 65% or at least about 64% or at least about 63% or at least about 62% or at least about 61% or at least about 60% or at least about 59% or at least abo ut 58% or at least about 57% or at least about 56% or at least about 55% or at least about 54% or at least about 53% or at least about 52% or at least about 51% or at least about 50% or at least about 49% or at least about 48% or at least about 47% or at least about 46% or at least about 45% or at least about 44% or at least about 43% or at least about 42% or at least about 41% or at least about 40% or at least about 39% or at least about 38% or at least about 37% or at least about 36% or at least about 35% or at least about 34% or at least about 33% or at least about 32% or at least about 31% or at least about 30% or at least about 29% or at least about 28% or at least about 27% or at least about 26% or at least about 25% or at least about 24% or at least about 23% or at least about 22% or at least about 21% or at least about 20% or at least about 19% or at least about 18% or at least about 17% or at least about 16% or at least about 15% or at least about 14% or at least about 13% or at least about 12% or at least about 11% or at least about 10% or at least about 9% or at least about 8% or at least about 7% or at least about 6% or at least about 5% or at least about 4% or at least about 3% or at least about 2% or at least about 1% of the cancer cell killing activity as a full dose of the non-caspase inhibitor anti-cancer agent would produce if used at full strength.

Another way of addressing this beneficial effect of the combination of the caspase inhibitor and the non-caspase inhibitor anti-cancer agent is to produce formulations that only kill (or not kill) at least about 99% or at least about 98% or at least about 97% or at least about 96% or at least about 95% or at least about 94% or at least about 93% or at least about 92% or at least about 91% or at least about 90% or at least about 89% or at least about 88% or at least about 87% or at least about 86% or at least about 85% or at least about 84% or at least about 83% or at least about 82% or at least about 81% or at least about 80% or at least about 79% or at least about 78% or at least about 77% or at least about 76% or at least about 75% or at least about 74% or at least about 73% or at least about 72% or at least about 71% or at least about 70% or at least about 69% or at least about 68% or at least about 67% or at least about 66% or at least about 65% or at least about 64% or at least about 63% or at least about 62% or at least about 61% or at least about 60% or at least about 59% or at least about 58% or at least about 57% or at least about 56% or at least about 55% or at least about 54% or at least about 53% or at least about 52% or at least about 51% or at least about 50% or at least about 49% or at least about 48% or at least about 47% or at least about 46% or at least about 45% or at least about 44% or at least about 43% or at least about 42% or at least about 41% or at least about 40% or at least about 39% or at least about 38% or at least about 37% or at least about 36% or at least about 35% or at least about 34% or at least about 33% or at least about 32% or at least about 31% or at least about 30% or at least about 29% or at least about 28% or at least about 27% or at least about 26% or at least about 25% or at least about 24% or at least about 23% or at least about 22% or at least about 21% or at least about 20% or at least about 19% or at least about 18% or at least about 17% or at least about 16% or at least about 15% or at least about 14% or at least about 13% or at least about 12% or at least about 11% or at least about 10% or at least about 9% or at least about 8% or at least about 7% or at least about 6% or at least about 5% or at least about 4% or at least about 3% or at least about 2% or at least about 1% of the non-cancer cells that are killed by the non-caspase inhibitor anti-cancer agent if it would be used alone to produce the same therapeutic effect.

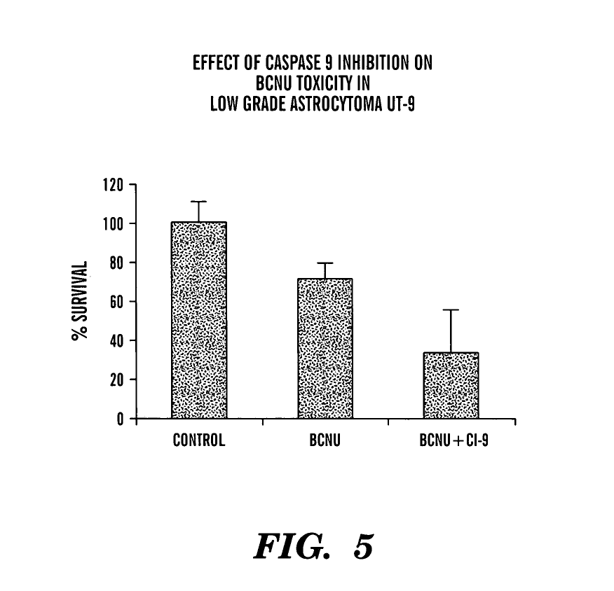

The paragraphs above address, without intention of being wholly inclusive, the ability of the disclosed compositions to reduce the amount of non-caspase inhibitor anti-cancer agent needed to get the same therapeutic effect, by addressing the reduced percent of the amount of non-caspase inhibitor anti-cancer agent that would be used alone. Alternatively, the amount of non-caspase inhibitor that can be used in the formulations can also be addressed by taking a percent of the killing activity of the non-caspase inhibitor anti-cancer agent obtained if used alone. For example, a formulation could contain an amount of non-caspase inhibitor anti-cancer agent that kills 50% of the cancer cells that a full dose of the same reagent would kill. For brevity each of the above variations and percent discussed above are not repeated here but are considered disclosed for this limitation. Therefore, it is understood that just as for the lists of different percentages related to “the reduced percent of the amount of non-caspase inhibitor anti-cancer agent that would be used alone” each and every disclosed percentage is also applicable in conjunction with a limitation related to the “percent of the killing activity of the non-caspase inhibitor anti-cancer agent obtained if it would be used alone.” The disclosed compositions and mixtures also are useful for enhancing the efficacy of an existing dose of a non-caspase inhibitor anti-cancer agent or anti-oxidant. Thus disclosed are combinations of caspase inhibitors and non-caspase inhibitor anti-cancer agents and/or anti-oxidants that enhance the tumor cell killing relative to the tumor cell killing of the non-caspase inhibitor anti-cancer agent or anti-oxidant if used alone or in combination (ie a combination of non-caspase inhibitor anti-cancer agent and anti-oxidant). The disclosed compositions and combinations can also decrease chemoresistance to non-caspase inhibitor anti-cancer agents and/or antioxidants. For example, FIG. 5 indicates the possibility of reversing chemoresistance through the co-application of caspase inhibitors and chemotherapeutic agents.

The paragraphs above also address the ability of the disclosed compositions to reduce the amount of non-caspase inhibitor anti-cancer agent needed to get the same therapeutic effect, by addressing the reduced percent of the amount of non-caspase inhibitor anti-cancer agent that would be used alone and by taking a percent of the killing activity of the non-caspase inhibitor anti-cancer agent obtained if it would be used alone. Alternatively, the amount of non-caspase inhibitor that can be used in the formulations can also be addressed by taking a percent of the non-cancer cells that are killed (or “not killed”) by disclosed combination formulation as compared to the non-caspase inhibitor anti-cancer agent if used alone. For example, a combined formulation could contain an amount of non-caspase inhibitor anti-cancer agent and caspase inhibitor that only “kills less than 50% of the non-cancer cells that a full dose of the non-caspase inhibitor anti-cancer reagent would kill if used alone”. For brevity each of the above variations and percent discussed above are not repeated here but are considered disclosed for this limitation. Therefore, it is understood that just as for the lists of different percentages related to “the reduced percent of the amount of non-caspase inhibitor anti-cancer agent that would be used alone” each and every disclosed percentage is also applicable in conjunction with a limitation related to the “kills less than 50% of the non-cancer cells that a full dose of the non-caspase inhibitor anti-cancer reagent would kill if used alone.”

(3) Antioxidant Formulations

Also disclosed herein are formulations that comprise antioxidant compositions. The formulations of caspase inhibitors for use in treating cancer and the formulations having caspase inhibitors and non-caspase inhibitor anti-cancer agents can be combined with antioxidant agents and administered for anti-cancer regimens. The addition of the antioxidant allows for decreased amount of either the caspase inhibitor or the non-caspase inhibitor anti-cancer agent, which as discussed herein can be beneficial. The anti-oxidant can also enhance the efficacy of other chemotherapy so as to increase the amount of tumor cells that are killed. The formulations containing the antioxidant can be addressed in each and every way as discussed herein for the combination formulations of caspase inhibitors and non-caspase inhibitor anti-cancer agents. In other words, the amount of the antioxidant in the formulation can be based on a percentage of that needed to be therapeutic alone, for both formulations containing the caspase inhibitor and formulations containing the caspase inhibitor and the non-caspase inhibitor anti-cancer agents. Likewise, the amount of the antioxidant can be addressed by looking at the percent of cancer cells killed and the percent of non-cancer cells killed (or not killed). Any antioxidant agent can be used in this regard, although it is currently indicated that combination of anti-oxidants will provide greater potency.

This needs to be linked out to our own anti-oxidant research and the vast existing body of literature on combining anti-oxidants and chemotherapeutic agents. One line of argument might be to ask whether caspase inhibition works by the same means as other means of enhancing chemotherapy (i.e., anti-oxidants).

c) Pharmaceutical Carriers/Delivery of Pharamceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the composition. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer. 60:215-281, (1989); Bagshawe, eta., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue: Hughes et al., Cancer Research, 49:6214-6220, (1989); and Litzinger and Huang, Biochimica et Biophysica Acta, 1104:179-187, (1992). In general, receptors are involved in pathways of endocytosis, either constitutive or ligand induced. These receptors cluster in clathrin-coated pits, enter the cell via clathri-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

(1) Pharmaceutically Acceptable Carriers

The compositions, including antibodies, can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether loal or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed compositions and combinations and mixtures can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, enmlsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

(2) Therapeutic Uses

The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

d) Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include a caspase inhibitor and a non-caspase inhibitor anti-cancer agent in formulations ready for delivery to a subject.

2. Methods of Making the Compositions

The compositions disclosed herein and the compositions necessary to perform the disclosed methods can be made using any method known to those of skill in the art for that particular reagent or compound unless otherwise specifically noted.

3. Methods of Using the Compositions

a) Methods of Using the Compositions as Research Tools

The disclosed compositions can be used in a variety of ways as research tools. For example, the disclosed compositions, such as the disclosed combinations, can be used to study apoptotic pathways.

b) Methods of Inhibiting Cancer Cell Proliferation

The disclosed compositions and formulations can be used to inhibit abberant cellular proliferation. For example, the disclosed compositions can be used to inhibit cell growth of cancer cells. This disclosed compositions can be used to inhibit cancer cell proliferation. Thus, the compositions can be used to treat patients with cancer. It is understood that any therapeutic effect can be beneficial and that a patient does not need to cured to be treated. The compositions can be used to kill cancer cells. The killing of a cancer cell means that the cell not only does not divide, it also gets destroyed. It can be beneficial to both inhibit the growth of a cancer cell as well as kill a cancer cell.

The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, glioblastomas, nephroblastoma, neuroblastomas, astrocytomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer. That this treatment protocol should be broadly applicable is supported by the large numbers of commonalities that have been seen in the response of divergent cancer cell populations to the same stimulus, and to the generalities of cancer cell behavior that have emerged from the study of the effects of oncogene cooperation, and multiple other lines of discovery as will be well known to those skilled in the arts.

Compounds disclosed herein may also be used for the treatment of precancer conditions such as cervical and anal dysplasias, other dysplasias, severe dysplasias, hyperplasias, atypical hyperplasias, and neoplasias.

c) Modifications

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example

### Killing of Tumor Cells, and Enhancement of Tumor Cell Killing by Cytotoxic Agents, by Application of Caspase Inhibitors

A variety of cells were exposed to BCNU (also known as carmustine) in the presence of a variety of caspase inhibitors. This alkylating agent is frequently employed in the treatment of cancers of the central nervous system, as well as for treatment of certain lymphomas. The growth of tumor cells and normal human brain precursor cells in chemically-defined medium was assayed under various conditions of alkylating agent and/or caspase inhibitor. Cells were exposed to BCNU at varying dosages, depending upon the outcome of characterization of their sensitivity to BCNU. In general, dosages were employed for which it would be possible to recognize protection from the cytotoxic effects of this alkylating agent, as well as to recognize increased anticancer activity depending on the conditions, of for example, the caspase inhibitor and the activity of this compound.

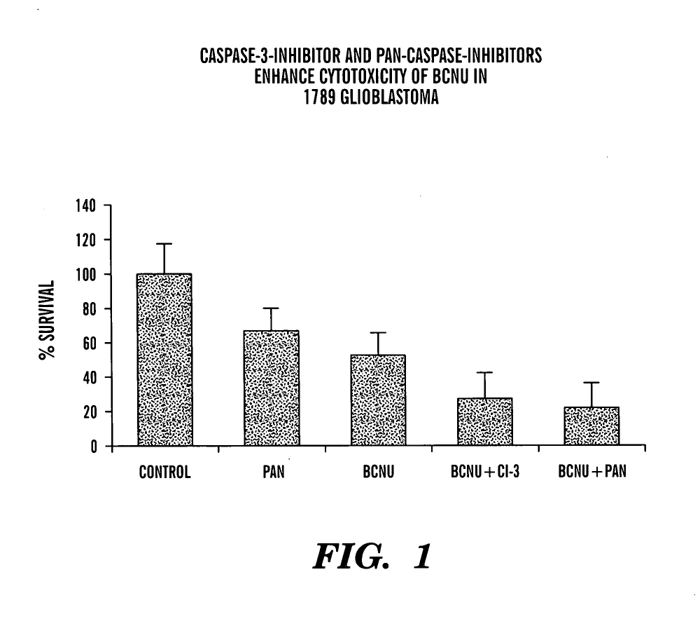

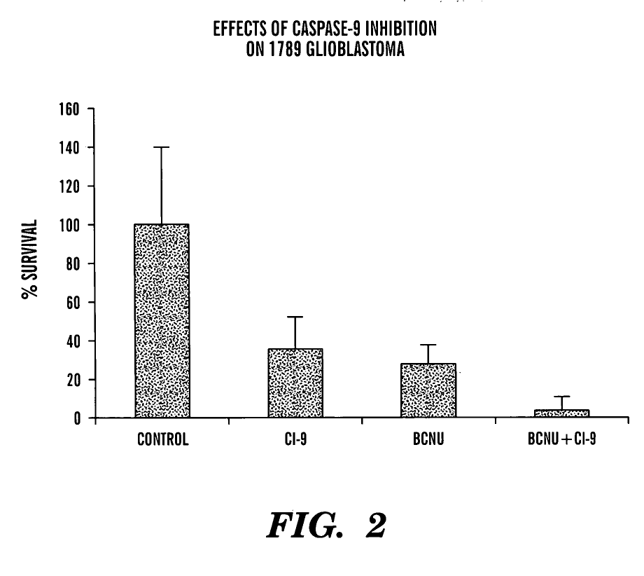

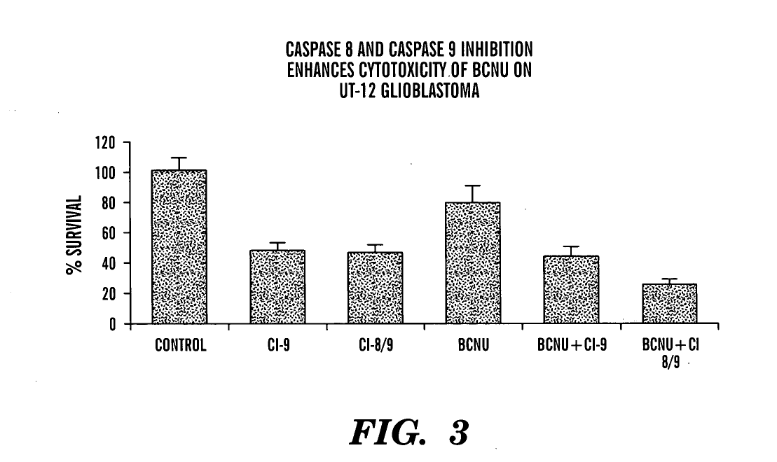

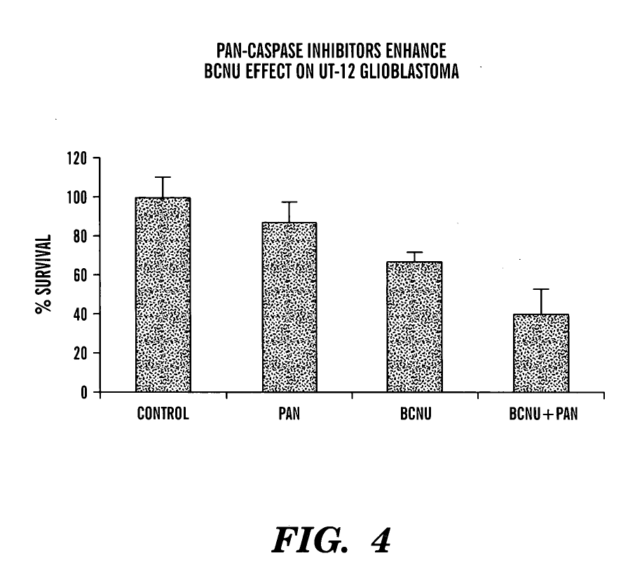

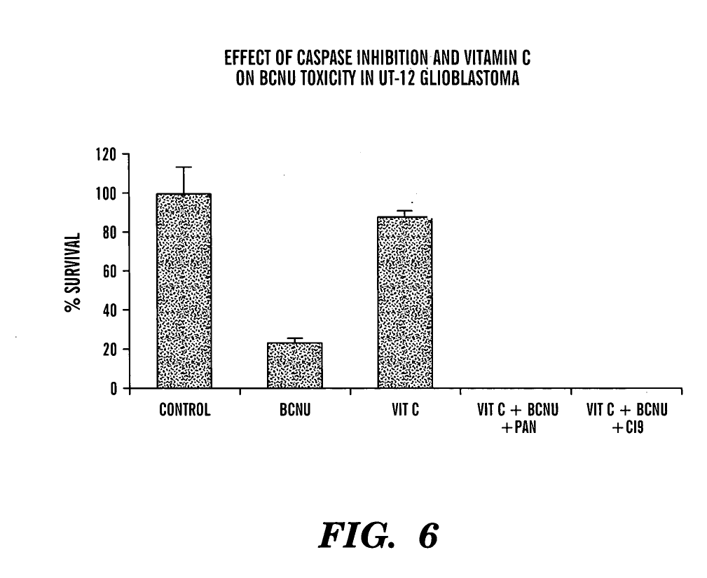

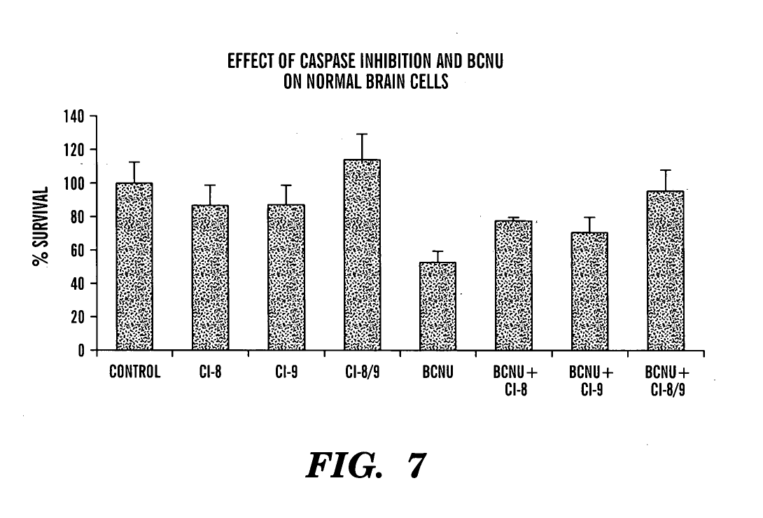

A variety of examples of the results obtained are shown in the FIGS. 1-7. The general protocol used to produce this data was as follows: Cells were plated at 1000 cells/well in 24-well plates. After 24 hours, cells were pretreated for 1 hour with caspase inhibitors at a concentration of 20 microM, following exposure to BCNU for 1 hour at concentrations that would kill approximately 50% of the tumor cells, as determined by dose-response experiments. In general, the BCNU concentration applied ranged from 5 microg/ml to 20 microg/ml. Exposure periods were based upon the known clearance rate of BCNU in vivo. 48 hours after BCNU treatment, cells were labeled with MTT and counterstained with DAPI, to determine the number of surviving cells. Percentages of cell survival were normalized to controls. All experiments were performed at least as quadruplicates. Error bars represent SEM.

The data herein show that inhibition of caspase activity has the effect of killing tumor cells and enhancing killing of tumor cells in conjunction with chemotherapy regimens. The data herein indicate that caspase inhibition without the aid of other chemotherapy agents inhibits cancer cell growth and causes cancer cell damage without damaging non-cancer cells and thus can be used as a therapeutic strategy alone in the treatment of cancer. The data disclosed herein also indicate that caspase inhibitors can also be combined with other cancer treatments to enhance the efficacy of the other cancer treatments and in certain applications the anti-cancer activity of the caspase inhibitor. The widespread importance of caspases in cellular function and the data herein indicate that co-application of caspase inhibitors with a large variety of different kinds of cancer therapies may enhance the effectiveness of those therapies. In addition, the data herein indicate that tumors not only are sensitive to pan caspases but also to specific caspases.

1. A composition comprising a caspase inhibitor and a non-caspase inhibitor anti-cancer agent.

2. The composition of claim 1, wherein the caspase inhibitor is a pan caspase inhibitor.

3. The composition of claim 2, wherein the caspase inhibitor is selected from the group consisting of inhibitors of caspase-9, caspase-3, caspase-8 or pan-caspase inhibitors.

4. The composition of claim 1, wherein the caspase inhibitor inhibits the production of a caspase.

5. The composition of claim 1, wherein the caspase inhibitor inhibits the activation of a caspase.

6. The composition of claim 1, wherein the caspase inhibitor inhibits a signaling pathway of a caspase.

7. The composition of claim 1, wherein the non-caspase anti-cancer agent is selected from the group consisting of alkylating agents, DNA strand breaking agents, antimetabolites, topoisomerase inhibitors, tubulin interactive agents, and mitotic inhibitors.

8. The composition of claim 1, wherein the non-caspase inhibitor anti-cancer agent is BCNU.

9. The composition of claim 1, further comprising an antioxidant.

10. The composition of claim 1, wherein the anti-cancer agent is itself an antioxidant.

11. The composition of claims 10, wherein the antioxidant is vitamin C or a glutathione pro-drug.

12. The composition of claim 1, further comprising a pharmaceutical carrier.

13. A method of inhibiting the growth of a cancer cell comprising introducing the composition of claim 1 to the cell.

14. The method of claim 1, wherein the cancer cell is killed.

15. A method of treating a subject having cancer comprising administering the composition of claim 12 to the subject.

16. A method of inhibiting the growth of a cancer cell comprising introducing a caspase inhibitor to the cell.

17. The method of claim 16, wherein the cancer cell is killed.

18. A method of treating a subject having cancer comprising administering a caspase inhibitor in a pharmaceutically acceptable form to the subject.

19. The method of claim 18, wherein the caspase inhibitor is a pan caspase inhibitor.

20. The method of claim 18, wherein the caspase inhibitor is specific for caspase-3, caspase-8 or caspase-9.

21. The method of claim 18, wherein the caspase inhibitor inhibits the production of a caspase.

22. The method of claim 18, wherein the caspase inhibitor inhibits the activation of a caspase.

23. The method of claim 18, wherein the caspase inhibitor inhibits a signaling pathway of a caspase.

24. A composition comprising a caspase inhibitor and an antioxidant.

25. The composition of claim 24, wherein the caspase inhibitor is a pan caspase inhibitor.

26. The composition of claim 24, wherein the caspase inhibitor is specific for a caspase selected from the group consisting of caspase-3, caspase-8 or caspase-9.

27. The composition of claim 24, wherein the caspase inhibitor inhibits the production of a caspase.

28. The composition of claim 24, wherein the caspase inhibitor inhibits the activation of a caspase.

29. The composition of claim 24, wherein the caspase inhibitor inhibits a signaling pathway of a caspase.

30. The composition of claim 24, wherein antioxidant is selected from the group consisting of non-flavonoid antioxidants, multi-carotenes, beta-carotenes, alpha- carotenes, gamma-carotenes, lycopene, lutein and zeanthins, selenium, Vitamin E, tocopherol, vitamin E succinate, trolox, Vitamin C, Niacin, Vitamin A, 13-cis retinoic acid, N-acetyl-L-cysteine, glutathione pro-drugs, sodium ascorbate, pyrrolidin-edithio-carbamate, coenzyme Q10, peroxidases, glutathione peroxidase, catalase, superoxide dismutase, glutathione transferase, glutathione reductase, glucose 6-phosphate dehydrogenase, glutathione, ceruloplasmin, cysteine, cysteamine, flavenoids, and mimetics, analogs and polymers thereof.

31. The composition of claim 24, wherein the antioxidant is vitamin C.

32. The composition of claim 1, further comprising a non-caspase anticancer agent.

33. (canceled)

34. The composition of claim 24, further comprising a pharmaceutical carrier.

35. A method of inhibiting the growth of a cancer cell comprising introducing the composition of claim 24 to the cell.

36. The method of claim 35, wherein the cancer cell is killed.

37. A method of treating a subject having cancer comprising administering the composition of claim 34 to the subject.
